(19) United States
(12) Patent Application Publication
(10) Pub. No.: US 2007/0219622 A1
(43) Pub. Date: Sep. 20, 2007

(54) Stent-graft structure having one or more stent pockets

(75) Inventor: Shyam SV Kuppurathanam, Bloomington, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 11/716,818

(22) Filed: Mar. 12, 2007

Related U.S. Application Data
(60) Provisional application No. 60/783,595, filed on Mar. 17, 2006.

(51) Int. Cl. A61F 2/06; A61F 2/86

(52) U.S. Cl. 623/1.13; 623/901

(57) ABSTRACT

A stent-graft assembly is provided for a variety of medical treatments. The stent-graft assembly comprises an inner graft, an outer graft, and at least one stent disposed circumferentially between the inner graft and outer graft. The inner graft is attached directly to the outer graft circumferentially at a first location proximal to a first stent, and further attached directly to the outer graft circumferentially at a second location distal to the first stent, thereby forming a first pocket that houses the first stent. Neither the inner graft nor the outer graft is attached directly to the stent, permitting improved stent flexibility and reducing manufacturing complexity.

### PRIORITY CLAIM

This invention claims the benefit of priority of U.S. Provisional Application Ser. No. 60/783,595, entitled “Stent-Graft Structure Having One or More Stent Pockets,” filed Mar. 17, 2006, the disclosure of which is hereby incorporated by reference in its entirety.

### BACKGROUND

The present invention relates generally to medical devices, and in particular, to a stent-graft having inner and outer graft layers and one or more stents disposed circumferentially therebetween.

Although stent-graft assemblies may be used to treat a number of medical conditions, one common use of stent-graft assemblies relates to the treatment of aneurysms. An aneurysm is an abnormal widening or ballooning of a portion of an artery, which may be caused by a weakness in the blood vessel wall. High blood pressure and atherosclerotic disease may also contribute to the formation of aneurysms. It is possible for aneurysms to form in blood vessels throughout the vasculature. Some common types of aneurysms include aortic aneurysms, cerebral aneurysms, popliteal artery aneurysms, mesenteric artery aneurysms, and splenic artery aneurysms. If not treated, an aneurysm may eventually rupture, resulting in internal hemorrhaging. In many cases, the internal bleeding is so massive that a patient can die within minutes of an aneurysm rupture. For example, in the case of aortic aneurysms, the survival rate after a rupture may be as low as 20%.

Traditionally, aneurysms have been treated with surgery. For example, in the case of an abdominal aortic aneurysm, the abdomen is opened surgically and the widened section of the aorta is removed. The remaining ends of the aorta are then surgically reconnected. In certain situations, the surgeon may choose to replace the excised section of the aorta with a graft material such as Dacron, instead of directly reconnecting the two ends of the blood vessel together. In still other situations, the surgeon may put a clip on the blood vessel at the neck of the aneurysm between the aneurysm and the primary passageway of the vessel. The clip then prevents blood flow from the vessel from entering the aneurysm.

An alternative to traditional surgery is endovascular treatment of the blood vessel with a stent-graft. This alternative involves implanting a stent-graft in the blood vessel across the aneurysm using conventional catheter-based placement techniques. The stent-graft treats the aneurysm by sealing the wall of the blood vessel with a generally impermeable graft material. Thus, the aneurysm is sealed off and blood flow is kept within the primary passageway of the blood vessel. Increasingly, treatments using stent-grafts are becoming preferred since the procedure may result in less trauma and a faster recuperation.

Although stent-grafts are frequently used for treating aneurysms, other medical treatments also use stent-grafts and still other uses are being explored. Additional applications for stent-grafts may be developed in the future. One example of other uses for stent-grafts is the surgical use of stent-grafts as artificial or replacement vessels. In the case of the vascular system, stent-grafts may be used to replace excised sections of diseased arteries with an artificial replacement vessel. Typically, this would be performed surgically by connecting the ends of the stent-graft to the ends of the artery remaining in the patient's body. Thus, in this application, the stent-graft acts like a blood vessel by directing blood flow through the lumen of the stent-graft and preventing blood flow through the walls of the stent-graft.

Stent-grafts may be used in still other applications as well. For example, stent-grafts may be used to treat stenosed arteries or other vascular conditions. Stent-grafts may also be used to treat a variety of non-vascular organs, such as the esophagus, trachea, colon, biliary tract, urinary tract, prostate and the brain.

One type of stent-graft currently known in the art is constructed with a stent disposed between inner and outer layers of graft material. The graft layers typically are secured to the stent in some manner. Various techniques for securing graft layers to a stent are currently known. However, the known conventional techniques have numerous problems associated therewith.

One technique for securing graft layers to a stent generally involves adhering the graft layers directly to the stent itself. This is normally accomplished by suturing the graft layers to the struts of the stent or some other part of the stent structure. However, this process must be done manually by specialists using special needles and forceps to sew thread through the graft material, around the struts of the stent, and finally knotting the ends of the thread. This is a very labor intensive task that is time consuming and expensive, thus raising the cost of stent-grafts made by this process.

Moreover, stent-grafts made by suturing the graft layers to the stent lose much of the flexibility inherent in the stent itself. This is generally caused by the direct attachment of the graft layers to the stent structure, which forces the entire assembly (i.e., both the graft layers and the stent) to move simultaneously together. As a result, the graft layers restrict the movement of the stent structure.

Flexibility of the assembled stent-graft is important for several reasons. For example, radial flexibility is important to allow the stent-graft to be collapsed onto a delivery system while also allowing the stent-graft to expand at the site of implantation. Axial flexibility is also important to enable the stent-graft to bend as it is guided through tortuous pathways to reach the site of implantation. Even after implantation, axial and radial flexibility remain important when the stent-graft is implanted in an area of the body that is expected to experience frequent movement. However, despite the importance of flexibility, stent-grafts that secure the graft layers directly to the stent are relatively inflexible compared to other types of stents.

Another technique that is used for securing graft layers to a stent generally involves encapsulating the stent or a portion thereof with an inner and an outer layer of graft material. In this type of stent-graft, the two layers of graft material are adhered to each other through open areas in the stent structure. Some additional bonding may also occur between each graft layer and the stent structure itself. Typically, the inner and outer graft layers are adhered by heating the graft layers or using adhesives. However, this type of stent-graft also lacks flexibility, as described above. This is due in general to the encapsulated construction of these stent-grafts. In particular, the areas in which the two graft layers are attached abut against the structure of the stent, thereby forcing the graft layers to move together with the stent. This causes the graft layers to restrict the movement of the stent structure. Thus, even when the graft layers are not directly secured to the stent as described, the graft layers are still unable to move independently of the stent.

### SUMMARY

In a first embodiment, the stent-graft assembly comprises an inner graft, an outer graft, and at least one stent disposed between the inner graft and the outer graft. The inner graft is attached directly to the outer graft circumferentially at a first location proximal to a first stent, and further attached directly to the outer graft circumferentially at a second location distal to the first stent, thereby forming a first pocket that houses the first stent. Neither the inner graft nor the outer graft is attached directly to the stent, permitting improved stent flexibility within the first pocket.

If desired, multiple stents may be employed. For example, a second stent may be disposed within a second pocket formed between the inner graft and the outer graft, the second pocket formed at a location distal to the first pocket. In this embodiment, the circumferential attachment of the inner graft to the outer graft separates the first pocket from the second pocket. If additional stents are employed, each adjacent stent pocket may be separated by circumferentially attaching the inner graft to the outer graft at additional locations.

A method of manufacturing a stent-graft also is provided. The method comprises providing an inner graft, an outer graft, and disposing the inner graft substantially within the outer graft to form an annular passage therebetween. A proximal end of the inner graft may be attached to the proximal end of the outer graft. A first stent then maybe inserted through a portion of the annular passage, and then the inner graft may be attached to the outer graft at a second attachment point, thereby forming a first pocket configured to house the first stent therein. Additional stents may be inserted through a portion of the annular passage, and additional attachment points may be formed to house the additional stents. The inner graft may be attached to the outer graft by circumferentially sewing the inner and outer grafts together, by thermal bonding, using adhesives, and so forth.

Other devices, methods, features and advantages of the invention will be, or will become, apparent to one with skill in the art upon examination of the following figures and detailed description. It is intended that all such additional devices, methods, features and advantages be within the scope of the invention, and be encompassed by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like reference numerals designate corresponding parts throughout the different views.

FIG. 1 is a side view of a stent-graft.

FIG. 2 is a cross-sectional view of the stent-graft of FIG. 1 taken along line A-A.

FIG. 3 is a side-sectional view of a portion of the stent-graft of FIG. 1.

FIGS. 4A-4F illustrate a method of manufacturing the stent-graft of FIGS. 1-3.

FIG. 5 is a side view of an alternative stent-graft.

### DETAILED DESCRIPTION OF THE PREFERRED EMBODIMENTS

In the present application, the term “proximal” refers to direction that is generally closer to a physician during a medical procedure, while the term “distal” refers to a direction that is generally closer to a heart during the medical procedure.

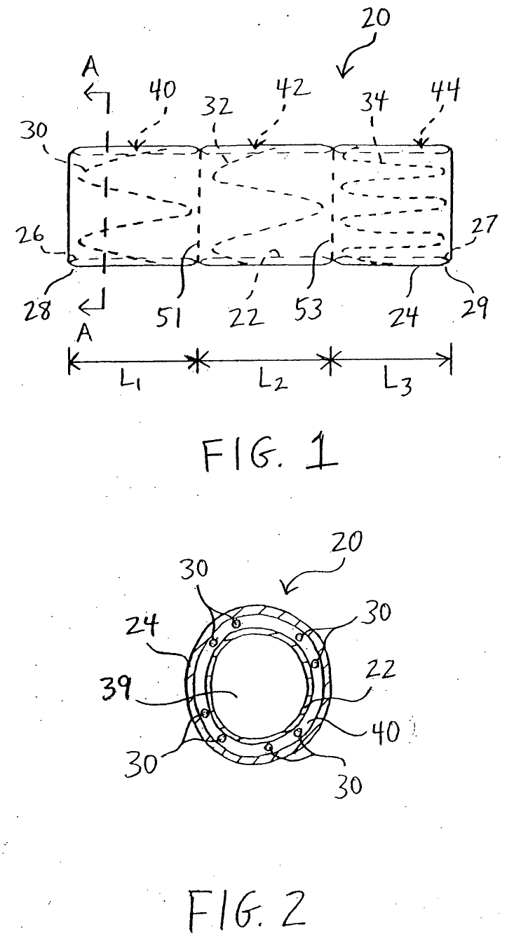

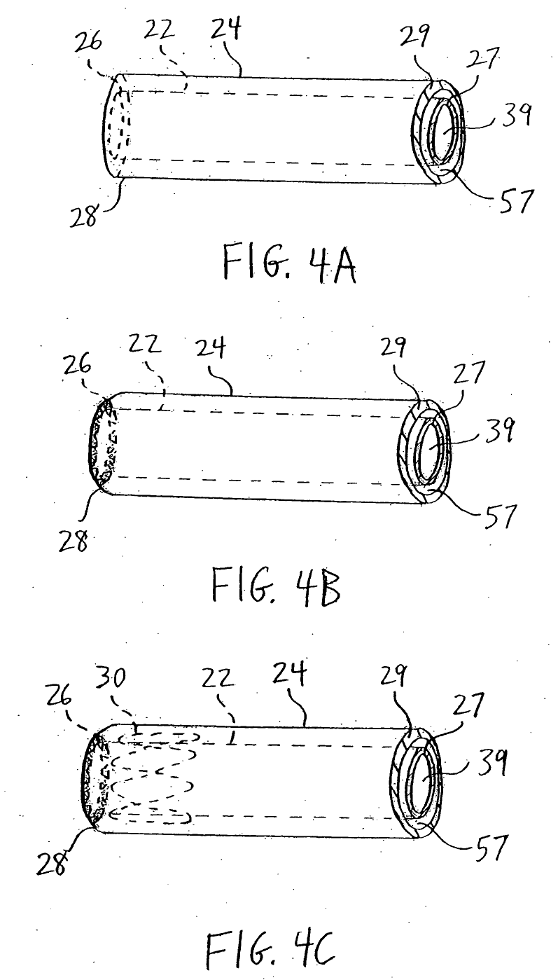

Referring now to FIGS. 1-3, a first stent-graft is described. Stent-graft 20 comprises inner graft 22 having proximal end 26 and distal end 27, and further comprises outer graft 24 having proximal end 28 and distal end 29, as shown in FIG. 1. Inner graft 22 has inner surface 60 and outer surface 61, while outer graft 24 has inner surface 70 and outer surface 71, as depicted in FIG. 3.

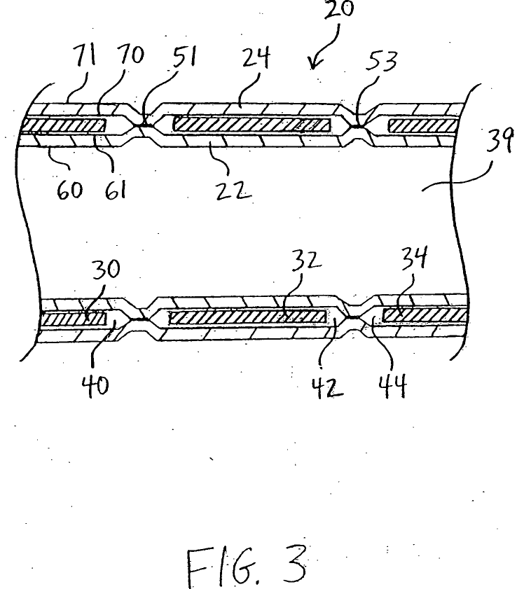

As shown in FIG. 3, first stent 30 is disposed between outer surface 61 of inner graft 22 and inner surface 70 of outer graft 24. First stent 30 is disposed within first pocket 40, which is formed between inner graft 22 and outer graft 24, as illustratively depicted in FIGS. 2-3.

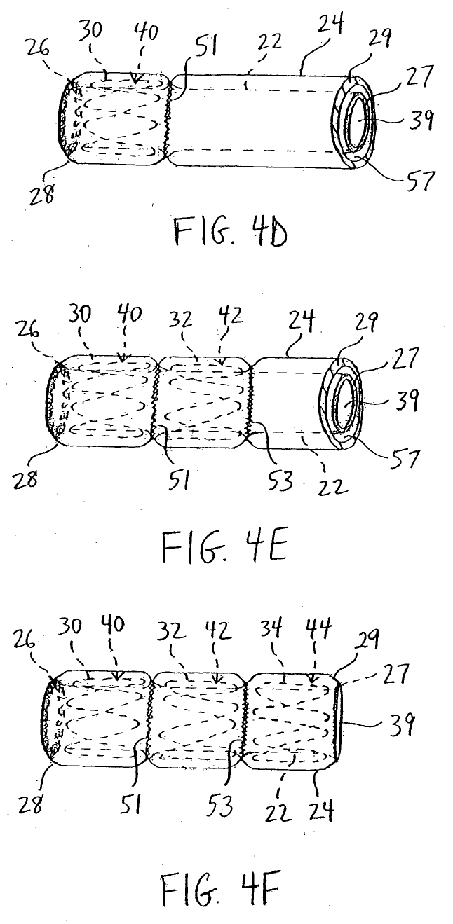

First pocket 40 preferably is formed by circumferentially attaching inner graft 22 to outer graft 24 at a first location proximal to first stent 30, and further circumferentially attaching inner graft 22 to outer graft 24 at a second location distal to first stent 30. In the embodiment of FIGS. 1-3, the proximal attachment location is formed where proximal end 26 of inner graft 22 is secured to proximal end 28 of outer graft 24, while the distal attachment location is formed at second attachment point 51, as depicted in FIG. 1, and described in greater detail below with respect to FIGS. 4A-4F. In effect, first stent 30 is moveably contained between the proximal ends of the two grafts and second attachment point 51.

In a preferred embodiment, multiple stents may be used. For example, in FIGS. 1-3, second stent 32 and third stent 34 are provided, although any number of stents may be employed. Second stent 32 is held within second pocket 42, which is disposed circumferentially between inner graft 22 and outer graft 24. As shown in FIG. 1, second pocket 42 may be formed between second attachment point 51 and third attachment point 53.

Similarly, third stent 34 is held within third pocket 44, which is disposed circumferentially between inner graft 22 and outer graft 24. Third pocket 44 may be formed between third attachment point 53 and a distal attachment location formed where distal end 27 of inner graft 22 is secured to distal end 29 of outer graft 24, as described in greater detail below with respect to FIGS. 4A-4F.

Several methods of securing together inner and outer grafts 22 and 24 are possible, depending on the particular needs of the application. For example, sutures made from polypropylene thread or other types of thread may be used to sew the inner and outer grafts together. Other examples of methods for securing together inner and outer grafts 22 and 24 include thermal bonding, such as welding or sintering, and the use of adhesives.

Various types of stents 30 may be used in conjunction with the present invention. For example, stents may be made from numerous metals and alloys, including stainless steel, nitinol, cobalt-chrome alloys, amorphous metals, tantalum, platinum, gold and titanium. Stents may also be made from non-metallic materials, such as thermoplastics and other polymers. The structure of the stent may also be formed in a variety of ways to provide a suitable intraluminal support structure. For example, stents may be made from a woven wire structure, a laser-cut cannula, individual interconnected rings, or any other type of stent structure that is known in the art. Regardless of the particular construction of the stent, it is usually desirable for the stent to be flexible in several directions, including both radial and axial flexibility. Stents may also be designed to be either balloon-expandable or self-expandable, depending on the particular application of the stent.

As depicted in FIG. 1, first stent 30, second stent 32 and third stent 34 generally comprise a zig-zag shape, i.e., formed from a single wire having a plurality of substantially straight segments and a plurality of bent segments disposed between the substantially straight segments. As will be apparent one skilled in the art, stents 30, 32 and 34 may alternatively comprise any number of shapes. For example, the stents may comprise a support structure having a pattern of interconnected struts. The arrangement, shape and size of the struts that are employed may vary depending on the geometry of the support structure that is used, and many variations are possible. In alternative embodiments, the stents of stent-graft 20 may comprise different shapes, e.g., first stent 30 may have a Z-shaped configuration, while second stent 32 may comprise a support structure having a pattern of interconnected struts.

Regardless of their configurations, first stent 30, second stent 32 and third stent 34 each have a reduced diameter delivery state in which stent-graft 20 may be advanced to a target location within a vessel, duct or other anatomical site. The stents further have expanded deployed states in which they may be configured to apply a radially outward force upon the vessel, duct or other target location, e.g., to maintain patency within a passageway. In the expanded state, fluid flow is allowed through central lumen 39 of stent-graft 20.

Many different types of graft materials may also be used for inner graft 22 and outer graft 24. Common examples of graft materials currently used include expandable polytetrafluoroethylene (ePTFE), polytetrafluoroethylene (PTFE), Dacron, polyester, fabrics and collagen. However, graft materials may be made from numerous other materials as well, including both synthetic polymers and natural tissues. One graft material that holds particular promise in certain applications is small intestine submucosa (SIS). As those in the art know, SIS material includes growth factors that encourage cell migration within the graft material, which eventually results in the migrated cells replacing the graft material with organized tissues.

In one embodiment of the present invention, inner graft 22 and outer graft 24 may be manufactured using different fabric materials, thereby providing inner and outer surfaces having different characteristics. Further, in certain applications, it may also be helpful to impregnate or coat inner graft 22 and/or outer graft 24 with various therapeutic drugs that are well-known to those in the art.

Further, inner and outer grafts 22 and 24 may be formed using a variety of techniques already known to the art. For example, as will be described in greater detail with respect to FIGS. 4A-4F below, two separate sheets of graft material may be rolled into tubes, one or more stents may be disposed between the two sheets of graft material, and the graft materials then are secured directly together at multiple circumferential locations. Alternatively, unitary tubes may also be formed using a mandrel or the like, which are then coaxially inserted into or drawn over stents 30, 32 and 34.

In alternative embodiments of the present invention, longitudinal lengths of the various pockets 40, 42 and 44 may be different. As shown in FIG. 1, first pocket 40 has longitudinal length L₁, while second pocket 42 comprises length L₂ and third pocket comprises length L₃. These lengths L₁-L₃ may be different, depending on the nature of the procedure. For example, if a proximal portion of stent-graft 20 is to be disposed within a straight portion of a vessel but a distal region is disposed in a tortuous portion of the vessel, then it may be desirable to manufacture smaller pockets that hold smaller stents near the distal region of the stent-graft. Alternatively, the stents themselves may have different properties, for example, third stent 34 may be relatively flexible while first stent 30 is relatively rigid, and so forth. For example, third stent 34 may have more bends that first and second stents 30 and 32, as shown in FIG. 1.

Referring now to FIGS. 4A-4F, a method of manufacturing stent-graft 20 is described. In FIG. 4A, inner graft 22 and outer graft 24 are provided. As shown, inner graft 22 has proximal end 26 and distal end 27, while outer graft 24 has proximal end 28 and distal end 29. In one embodiment, inner and outer grafts 22 and 24 are formed from two separate sheets of graft material that are rolled into tubes, as depicted in FIG. 4A. Inner graft 22 has an outer diameter that is smaller than an inner diameter of outer graft 24, thereby allowing inner graft 22 to be disposed concentrically within outer graft 24. Annular passage 57 is formed between inner graft 22 and outer graft 24, as shown in FIG. 4A.

Proximal end 26 of inner graft 22 then is attached to proximal end 28 of outer graft 24, as shown in FIG. 4B. As discussed above, sutures made from polypropylene thread or other types of thread may be used to sew inner and outer grafts 22 and 24 together, or alternatively, the inner and outer grafts may be secured together using thermal bonding, such as welding or sintering, the use of adhesives, and so forth.

In a next step, shown in FIG. 4C, first stent 30 is inserted into annular passage 57 and is advanced in a distal to proximal direction towards the attached proximal ends of inner and outer grafts 22 and 24. The advancement of first stent 30 in a proximal direction may be performed manually or using a machine. As shown in FIG. 4C, first stent 30 is disposed just distal to the attached proximal ends 26 and 28 of inner and outer grafts 22 and 24, respectively.

Subsequently, inner and outer grafts 22 and 24 are circumferentially attached together at second attachment point 51, which is just distal to first stent 30, as shown in FIG. 4D. The coupling at second attachment point 51 may be achieved using any of the techniques described above. In effect, first pocket 40 is formed to hold first stent 30 between the attached proximal ends of the grafts and second attachment point 51. Since the graft materials are not directly attached to first stent 30, the stent is free to move within pocket 40 as needed during delivery and/or expansion of the stent.

If multiple stents are employed, then in a next step, second stent 32 is inserted into annular passage 57 and is advanced in a distal to proximal direction towards second attachment point 51. As shown in FIG. 4E, second stent 30 is disposed just distal to second attachment point 51 between inner and outer grafts 22 and 24. Then, another circumferential attachment is made between inner graft 22 and outer graft 24 at third attachment point 53 to form second pocket 42. In effect, second stent 32 is held within second pocket 42 at a location distal to second attachment point 51 and proximal to third attachment point 53.

Finally, third stent 34 is inserted into annular passage 57 and is advanced in a distal to proximal direction towards third attachment point 53. A final circumferential attachment is made between distal end 27 of inner graft 22 and distal end 29 of outer graft 24, thereby forming third pocket 44 between third attachment point 53 and the distal ends of the grafts, as shown in FIG. 4F. As will be apparent, if additional stents are used, then additional lengths of graft material are employed, and subsequent attachments between inner graft 22 and outer graft 24 may be made in the manner described above.

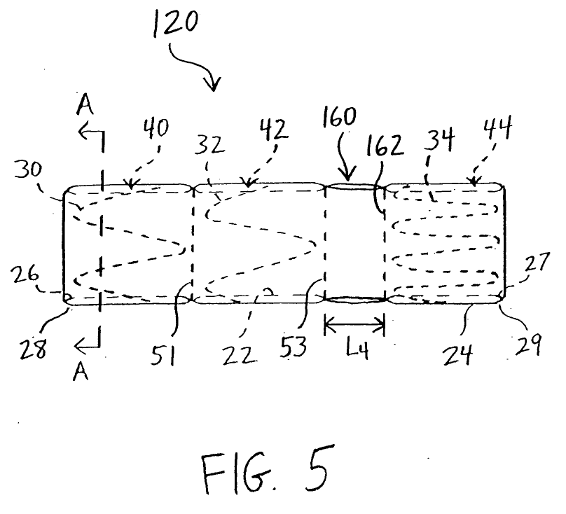

Referring now to FIG. 5, alternative stent-graft 120 is similar to stent-graft 20 of FIGS. 1-4, with a main exception that spacing section 160 is provided. Spacing section 160, which does not house a stent, is formed between second pocket 42 and third pocket 44. As shown in FIG. 5, spacing section 160 is formed between third attachment point 53, which encloses the distal end of stent 32, and spacing attachment point 162, which encloses the proximal end of stent 34. In effect, an additional attachment point is provided to form an empty space, i.e., without a stent, along a portion of the length of stent-graft 120.

Advantageously, spacing section 160 may permit flexibility along the longitudinal length of stent-graft 120. For example, since no stent is disposed in section 160, this section may be more axially flexible than portions of the stent-graft in which stents are housed. Thus, section 160 may axially flex, or pivot, as necessary to conform to an anatomical lumen. Length L₄ of spacing section 160 may be varied according to the needs of a procedure. As will be apparent, multiple spacing sections may be employed, e.g., between first pocket 40 and second pocket 42, and/or between second pocket 42 and third pocket 44 as shown.

Using the techniques of the present invention, stent-grafts 20 and 120 may be easier to manufacture and may be less expensive than traditional stent-grafts where the graft material is secured directly to the stent struts. The reason for this is that the graft layers are secured directly together instead of being secured to the structure of the stent. This avoids the difficulty of threading sutures around the stent struts, and the labor required may be less than traditional suturing techniques. Moreover, the labor required to secure inner graft 22 to outer graft 24 may be reduced even further if thermal bonding or adhesives are used to secure the graft layers together.

Another advantage associated with stent-grafts 20 and 120 is increased radial and axial flexibility compared to stent-graft assemblies having graft layers secured directly to the stent structure or stent-graft assemblies with graft material encapsulated onto the stent structure. Previous methods of securing graft materials to a stent structure restrict the movement of the graft material relative to the stent. Thus, conventional stent-graft assemblies are considerably less flexible than the underlying stents themselves. By contrast, stent-grafts 20 and 120 of the present invention form a series of pockets that permit associated stents to be housed therein, and permit the inner and outer grafts to move relative to the stents, particularly during flexure or expansion of the stents.

Stent-grafts 20 and 120 may be used in a number of medical applications for a variety of purposes. For example, stent-grafts 20 and 120 may be constructed with inner and outer grafts 22 and 24 made from SIS material. Although the SIS graft layers may be secured together with sutures, thermal bonding may also be used to avoid the introduction of foreign materials into the stent-graft. This may produce a stent-graft that is well-suited for replacement vessel applications, since the SIS material tends to become remodeled into the surrounding tissues after implantation.

While various embodiments of the invention have been described, it will be apparent to those of ordinary skill in the art that many more embodiments and implementations are possible within the scope of the invention. Moreover, the advantages described herein are only some of the advantages that may be possible with the invention and not all advantages will necessarily be achieved with every embodiment of the invention. Accordingly, the invention is not to be restricted except in light of the attached claims and their equivalents.

I claim:

1. A stent-graft assembly, comprising:
an inner graft having proximal and distal ends and inner and outer surfaces;
an outer graft having proximal and distal ends and inner and outer surfaces, wherein the outer surface of the inner graft is disposed substantially within the inner surface of the outer graft;
a first stent disposed within a first pocket formed between the outer surface of the inner graft and the inner surface of the outer graft; and
a second stent disposed within a second pocket formed between the outer surface of the inner graft and the inner surface of the outer graft, the second pocket formed at a location distal to the first pocket,
wherein the first stent and the second stent have different characteristics.

2. The stent-graft assembly of claim 1, wherein the first stent and the second stent have different material property characteristics.

3. The stent-graft assembly of claim 1, wherein the first stent and the second stent have different structural characteristics.

4. The stent-graft assembly of claim 3, wherein the first stent and the second stent have different axial flexibilities.

5. The stent-graft assembly of claim 1 wherein the inner graft is attached to the outer graft along at least a portion of a circumference thereof at a first location proximal to the first stent, and further attached to the outer graft at a second location distal to the first stent to form the first pocket, wherein the first pocket permits movement of the first stent therein.

6. The stent-graft assembly of claim 5, wherein the attached second location separates the first pocket from the second pocket.

7. The stent-graft assembly of claim 5, wherein the inner graft is attached to the outer graft by circumferentially sewing the inner and outer grafts together.

8. The stent-graft assembly of claim 5, wherein the inner graft is attached to the outer graft by thermal bonding.

9. The stent-graft assembly of claim 5, wherein the inner graft is attached to the outer graft using adhesives.

10. The stent-graft assembly of claim 1 further comprising a spacing section having a length disposed between the first pocket and the second pocket, wherein the spacing section does not comprise a stent along its length.

11. The stent-graft assembly of claim 1, wherein the first pocket and the second pocket have different longitudinal lengths.

12. The stent-graft assembly of claim 1, wherein the inner graft and the outer graft are manufactured using different fabric materials.

13. A method of manufacturing a stent-graft, the method comprising:
providing an inner graft having proximal and distal ends and an outer graft having proximal and distal ends, the inner graft having an outer diameter that is smaller than an inner diameter of the outer graft;
disposing the inner graft substantially within the outer graft to form an annular passage therebetween;
attaching the proximal end of the inner graft to the proximal end of the outer graft;
inserting a first stent through a portion of the annular passage; and
attaching the inner graft to the outer graft at a second attachment point, thereby forming a first pocket configured to house the first stent therein.

14. A stent-graft assembly, comprising:
an inner graft having proximal and distal ends and inner and outer surfaces;
an outer graft having proximal and distal ends and inner and outer surfaces, wherein the outer surface of the inner graft is disposed substantially within the inner surface of the outer graft;
a first stent disposed within a first pocket formed between the outer surface of the inner graft and the inner surface of the outer graft;
a second stent disposed within a second pocket formed between the outer surface of the inner graft and the inner surface of the outer graft, the second pocket formed at a location distal to the first pocket; and
a spacing section having a length disposed between the first pocket and the second pocket, wherein the spacing section does not comprise a stent along its length.

15. The stent-graft assembly of claim 14, wherein the inner graft is attached to the outer graft by circumferentially sewing the inner and outer grafts together.

16. The stent-graft assembly of claim 14, wherein the first stent and the second stent have different characteristics.

17. The stent-graft assembly of claim 16, wherein the first stent and the second stent have different structural characteristics.

18. The stent-graft assembly of claim 16, wherein the first stent and the second stent have different material property characteristics.

19. The stent-graft assembly of claim 14, wherein the inner graft and the outer graft comprise different fabrics.

20. The stent-graft assembly of claim 14, wherein the first pocket and the second pocket have different longitudinal lengths.
